(19) United States
(12) Patent Application Publication
(10) Pub. No.: US 2003/0197068 A1
(43) Pub. Date: Oct. 23, 2003

(54) Nebulizer chamber with vertical exit and anti-spill system

(76) Inventor: Riccardo Abate, (US)

Correspondence Address: MCGLEW & TUTTLE, PC, SCARBOROUGH STATION, SCARBOROUGH, NY, 10510, US

(21) Appl. No.: 10/414,141

(22) Filed: Apr. 15, 2003

(30) Foreign Application Priority Data
Apr. 17, 2002 (IT) BS2002U000044

(51) Int. Cl. A61M 11/06

(52) U.S. Cl. 239/338

(57) ABSTRACT

The invention concerns a nebulizer chamber for aerosol systems, which is made up of an outlet duct (21) which extends vertically to the top starting from an annular nebulised liquid passage, and an anti-spill system (24, 25) positioned between the outlet duct and the annular passage to prevent spilling of the medicinal liquid due to tilting or dropping of the chamber. The chamber also has a nebulizer nozzle with a baffle (30) to deflect the larger nebulised particles.

### FIELD OF THE INVENTION

This invention concerns in general the medicinal aerosol therapy atomizer equipment, and refers in particular to nebulizer chamber to be used in association with such equipment.

### PRIOR ART

Various chamber devices and configurations for aerosol treatment using liquid medication housing nebulizer pisper are already widely known.

According to the regulations in force in the sector, a nebulizerchamber for the above use must be such as not to permit accidental spilling of the medication liquid in the case of excessive angulations, dropping or turning the chamber itself upside-down.

A nebulizer chamber in a recent embodiment of an atomizer equipment has been designed with an anti-spill system which complies with the regulations for the sector, but it applies to a chamber with a lateral exit of the atomized liquid, therefore in the presence of a particular, specific configuration of the chamber body.

However, also well known are those nebulizer chambers whose body has a top vertically oriented outlet of the atomized liquid, that is parallel to the geometric axis of the body itself.

### OBJECT AND SUMMARY OF THE INVENTION

One objective of this invention is to equip these nebulizer chambers with vertical exit of atomized liquid with an efficient anti-spill system, also.

Another objective of the invention is to provide a nebulizer chamber, either with vertical or lateral exits, updated as regards to the pisper to improve its efficiency.

These objectives and implicit advantages are achieved with a nebulizer chamber according to claim 1 and with the particular characteristics in accordance with the claims (2.7).

### BRIEF DESCRIPTION OF THE DRAWINGS

Greater detail of the invention will become clear from the continuation of this description made with reference to the enclosed indicative and non-limiting drawings, in which:

FIG. 1 shows a view in perspective of a chamber with a top exit,

FIG. 2 shows a vertical cross-section of the chamber in FIG. 1 complete with a anti-spill system and revised pisper;

FIG. 3 shows a component, the one at the top, of the chamber in FIG. 1;

FIG. 4 shows a view from the bottom of the component shown in FIG. 3;

FIG. 5 shows a view in perspective of a chamber with lateral exit;

FIG. 6 shows a vertical cross-section of the chamber in FIG. 1;

FIG. 7 shows a cross-section of the chamber in FIG. 6 in direction of the arrows A-A;

FIG. 8 shows a view in perspective of the pisper for nebulizer chambers;

FIGS. 9, 10 and 11 show, respectively, front, side and top views of pisper in FIG. 8.

### DETAILED DESCRIPTION OF THE INVENTION

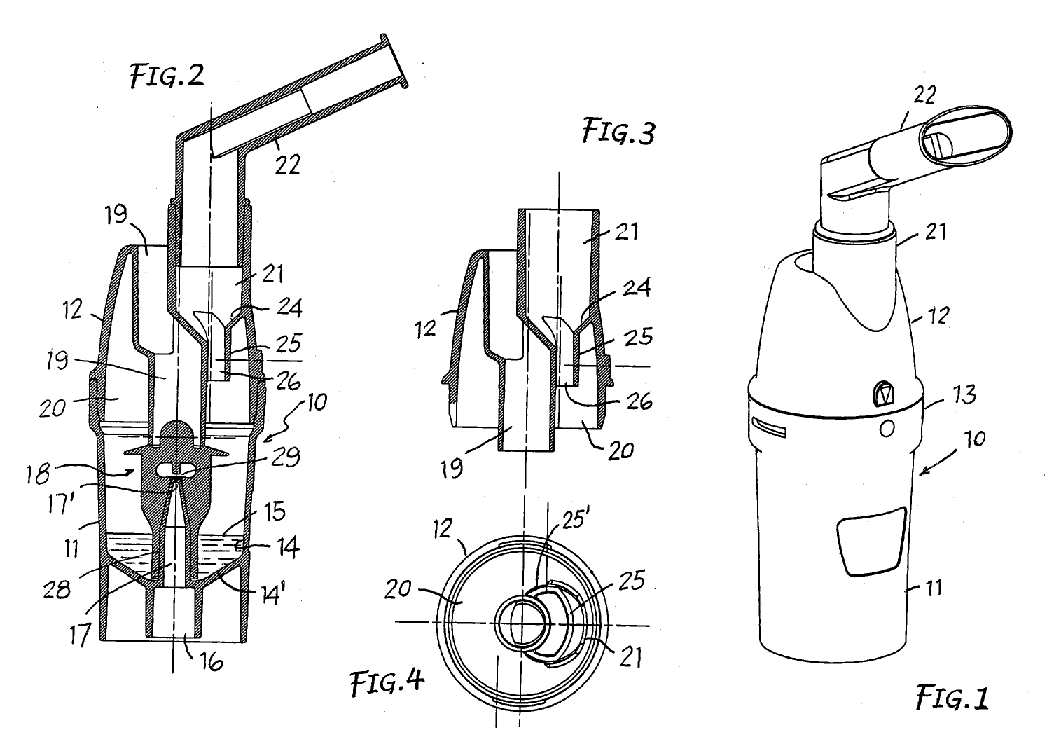

In the device shown in FIGS. 1 and 2, the chamber has a body 10 made up of a bottom element 11 and a top element 12, which are connected to each other, so as not to be separable, along a joining plane using, for example a threaded, or bayoneted or similar fit 13.

The lower element 11 forms a chamber 14 to contain a medicine to be nebulised 15 and having a bottom 14′ with a duct 16 for nebulising air input from a compressor—not shown.

The air conduct 16 has a section 17 which extends from the bottom 14′ of the chamber above the level of the medicinal liquid 15 and ends in a conical portion having a top orifice 17′. A nebulizer nozzle 18, also known as a “pisper”, is fitted to the extension of the air duct 16, the structure of which will be described later. The joining plane of the two components 11, 12 is above the pisper.

The top element 12 defining the body of the chamber 10 has a through conduit 19 extending from the bottom towards the top, and around this, an annular passage 20 closed at the top. The through conduit 19 is open at the bottom towards the nebulizer nozzle 18, whereas at the top it can be open or tapped to reduce the distance, according to needs. The annular passage 20 is required to make the nebulised liquid to rise up from the nebulizer nozzle 18 and is in communication with a vertical outlet duct 21 of the nebulised liquid at the top of the body 10. The outlet duct 21 can be connected to a mask or a mouthpiece 22, as shown in the drawings, or a pediatrics or adults nozzle as required, which if turned can act as a means for blocking the through passage 19.

The chamber described above is also equipped with an anti-spill system between the annular passage 20 and the outlet duct 21. The anti-spill system consists of a dividing baffle 24 which starting from an area of the wall called the outlet duct 21, from the side of the latter which is furthest from the through passage, extends towards the centre of the passage itself and continues towards the bottom of the annular duct 20 with a section 25 practically parallel to the external surface of the wall of the through passage. The vertical opposite edges of the section 25 of the dividing baffle 24 meet with the external surface of the wall of the through passage 19 as shown in FIGS. 3 and 4.

The dividing baffle 24 and its section 25 extend downwards, into the annular duct forming, on one side, with the adjacent external surfaces of the wall of the through passage 19, a passage 26 for the nebulised liquid towards the outlet 21 and, on the other side, a barrier which prevents the medicinal liquid from flowing out of said outlet if and when the chamber is tilted or lying flat.

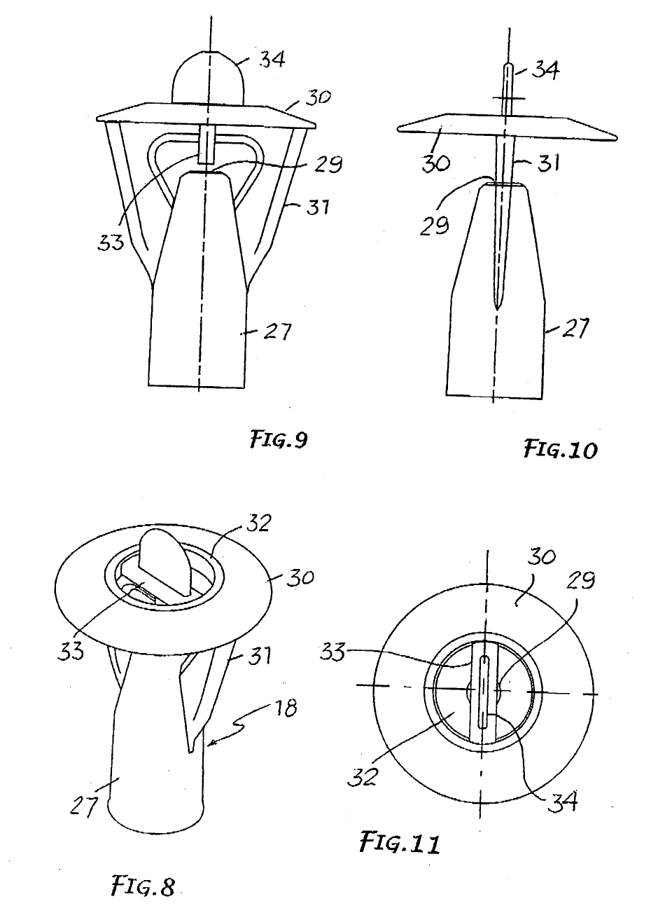

The nebulizer nozzle or pisper 18 referred to above—FIGS. 8-11—has the shape of a hood 27 which fits complementarily with the part 17 of the air duct 16 extending upwards in the chamber 14 and which forms with said part 17 an annular hollow space 28. The latter is open at the bottom towards the chamber 14 dipping into the medicine it contains and, at the top, flowing into an injector nozzle 29 placed above and in line with the orifice 17′.

Above the hood 27 there is a deflector plate 30 supported by at least two arms 31 having a centre hole 32 in line with the through passage 19 and placed crosswise in this area there is a flow breaker crosspiece 33 (pisper) above the injector nozzle 29 and possibly equipped with a grip lug 34.

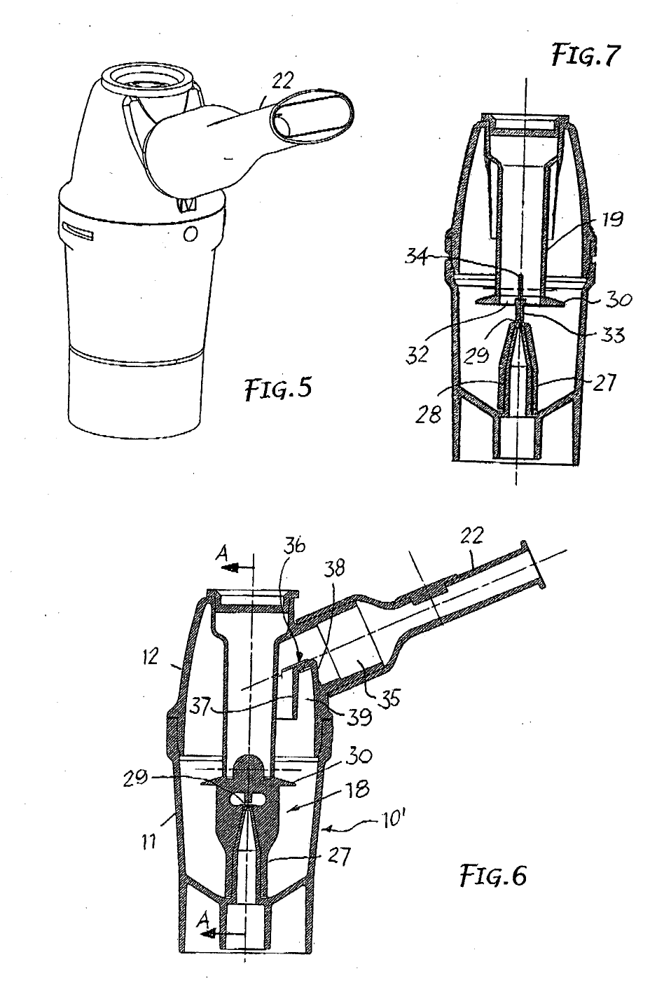

The nebulizer nozzle or pisper 18 positioned in this way can be applied both in a chamber with an vertical outlet passage 21 as in FIGS. 1 and 2, and in a chamber with a lateral outlet passage as shown in FIGS. 5 and 7 where the same reference numbers have been used to indicate the parts which are the same or equivalent as those described when referring to FIGS. 1-4.

This other chamber 10′ advantageously houses, on a level with its lateral outlet passage 35, a division 36 for two functions aimed at improving the use of the chamber: a first anti-spill function and a second to prevent the flow of saliva from the passage itself to the chamber containing the medicinal liquid.

The division 36 consists of a baffle positioned around and parallel to a part of the central duct 19 for air input, between the latter and the external wall of the top element 12, and it links up to a barrier 38 which projects from the lowest part of the outlet duct 35.

The baffle 37 extends downwards into the chamber 20, its opposite vertical edges fit against the wall of the central passage 19 and form together with the external wall of the top body 12 a pocket 39 to receive and retain the medicinal liquid stopping it from flowing out should the chamber be tilted or lying flat.

On the other side the barrier 38 blocks the lowest part of the outlet passage of the nebulised liquid preventing the backflow of saliva into the chamber and its unwanted mixing with the medicinal liquid.

In both cases, the help of the air which arrives from below to the passage 16 and enters the through passage 19 from the top, causes the medicinal liquid to be sucked through the annular hollow passage 28. The medicine is then made to flow towards the top part pf the nebulizer nozzle to reach the orifices 17′, 29 where air and medicine mix nebulising thanks to the obstacle in the form of a crosspiece 33.

The nebulised liquid passes radially under the deflector plate 30 from where the finest particles rise in the annular duct 20 and from here to the outlet passage 21 to be used, whereas the larger particles drop and collect in the chamber 14.

1. A nebulizer chamber for aerosol devices comprising a body (10) composed with two assembled elements (11,12) and forming:
a bottom chamber (14) for a medicinal liquid to be nebulised;
a duct (16) extending upwards from a bottom of said chamber, designed for the entrance of a flow of nebulising air having a top orifice (17′) and supporting a nebulizer nozzle (18);
a through passage (19) for air entrance, open both at the bottom towards the nebulizer nozzle (18) and at the top which if needed can be metered; and
an annular duct (20) around the through duct and protruding from said bottom chamber up to the outlet duct (21) of the nebulised liquid towards the user;
characterized by the fact that the outlet duct (21) extends vertically to the top of said annular duct (20) beside the through duct (19) and that a anti-spill system (24, 25) is placed between the vertical outlet duct and the annular duct so as to prevent spilling of the medicinal liquid from said outlet duct following tilting or dropping of the chamber.

2. Nebulizer chamber according to claim 1, wherein the anti-spill system includes a dividing baffle (24) reaching towards the centre of the outlet duct (21) starting from the wall of the duct itself furthest from the through duct, where said plate extends downwards into the annular duct (20) with a section (25) parallel to and not touching the wall of the through duct (19) so as to form together with said wall a passage for the nebulised liquid to flow from said annular duct to the vertical outlet duct.

3. Nebulizer chamber according to claim 2, wherein the edges of said section (25), an extension of said dividing plate (24) are in contact with the wall of the through duct (19) to form said passage for the nebulised liquid, the dividing plate forming a wall to stop the medicinal liquid entering the outlet duct (21).

4. Nebulizer nozzle (18) for nebulizer chambers used in aerosol systems, made up of a hood shaped element (27) terminating in an injector nozzle at the top (29) and having a central hole (32), and a flow breaker crosspiece (33) (pisper) placed above said injector nozzle, in the central hole of said deflecting plate (baffle).

5. Nebulizer nozzle according to claim 4, applicable in a chamber according to claims 1-3, where said hood shaped element (27) is associated with the part of the duct (16) for air flowing into the chamber (14) for the medicinal liquid so as to form with said part of said duct a hollow annular passage (28) dipping into the medicinal liquid in said chamber, and so that the top injector nozzle (29) is in line with the top orifice of said upward protruding part of said air duct and that the centre hole of the deflecting plate (30) (baffle) coincides with the through passage (19) of the chamber.

6. Nebulizer nozzle according to claim 4 applicable to a nebulizer chamber which is made up of a body (10) with two assembled elements (11, 12) forming:
a bottom chamber (14) for a medicinal liquid to be nebulised;
a duct (16) extending upwards in said chamber, used for the entrance of a flow of nebulising air having a top orifice (17′) and supporting a nebulizer nozzle (18);
a through passage (19) open both at the bottom towards the nebulizer nozzle (18) and at the top which if needed can be metered; and
an annular duct (20) around the through duct and protruding from said bottom chamber up to the outlet duct (21) of the nebulised liquid towards the user;

7. A chamber according to claim 6, where the slanting outlet duct (35) is associated with a division (36) having the dual function of anti-spill to stop medicinal liquid spilling out of the outlet duct caused by the chamber being dropped or held at a slant and as a barrier to prevent the flow of saliva into the medicinal liquid chamber.
